(19) United States
(12) Patent Application Publication
(10) Pub. No.: US 2010/0028199 A1
(43) Pub. Date: Feb. 4, 2010

(54) PROCESS FOR IMPROVING THE SMELL OF ELEMENTAL SULPHUR

(76) Inventors: Arnab Chatterjee, Amsterdam (NL); Carolus Matthias Anna Maria Mesters, Amsterdam (NL); Ronald Jan Schoonebeek, Amsterdam (NL)

Correspondence Address: SHELL OIL COMPANY, P O BOX 2463, HOUSTON, TX, 772522463, US

(21) Appl. No.: 12/444,226

(22) PCT Filed: Oct. 3, 2007
(86) PCT No.: PCT/EP07/60491

§ 371 Date: Jun. 2, 2009

(30) Foreign Application Priority Data
Oct. 5, 2006 (EP) 06121787.3

(51) Int. Cl. A61L 9/00

(52) U.S. Cl. 422/5

(57) ABSTRACT

A process for improving the smell of a stream of elemental sulphur comprising an organic polysulphide and/or a thiol, the process comprising the following step: (a) heating the stream of liquid elemental sulphur comprising an organic polysulphide and/or a thiol at a heating temperature in the range of from 360 to 700° C., at a pressure that is sufficient to maintain a liquid elemental sulphur phase to obtain heat-treated elemental sulphur.

### FIELD OF THE INVENTION

The invention provides a process for improving the smell of a stream of elemental sulphur comprising an organic polysulphide and/or a thiol.

### BACKGROUND OF THE INVENTION

Elemental sulphur is a by-product of oil and gas refining processes. It is known that elemental sulphur obtained as a by-product of oil and gas refining may be used as raw material for sulphuric acid or as a binder in sulphur cement or in other sulphur cement products, for example sulphur cement-aggregate composites like sulphur mortar, sulphur concrete or sulphur-extended asphalt.

In refineries, sulphur compounds in liquid hydrocarbonaceous streams are typically converted by reaction with hydrogen into hydrogen sulphide. Thus, a gaseous stream comprising hydrogen sulphide and hydrogen is obtained. The hydrogen sulphide separated from this gaseous stream or hydrogen sulphide separated from natural gas is typically converted into elemental sulphur. A well-known example of such process is the so-called Claus process.

Conversion of hydrogen sulphide into elemental sulphur using the Claus process has certain disadvantages. The oxidation step in the Claus process is not selective for hydrogen sulphide, therefore separation of the hydrogen sulphide from the remainder of the gas stream is necessary. In view of thermodynamic limitations, no complete conversion of hydrogen sulphide in a single process stage can be obtained.

An alternative for the Claus process is selective oxidation of hydrogen sulphide comprised in a hydrocarbonaceous gas stream. Selective oxidation processes are disclosed in for example U.S. Pat. No. 4,886,649, U.S. Pat. No. 4,311,683, U.S. Pat. No. 6,207,127 and WO2005/030638. Compared to the Claus process, selective oxidation has several advantages. An advantage is that a high conversion of hydrogen sulphide can be obtained in a single process stage. Another advantage is that the oxidation is selective for hydrogen sulphide, thus avoiding the need for separation of hydrogen sulphide from the other gas components.

In WO2005/030638 is disclosed a process for the selective oxidation of hydrogen sulphide by contacting a hydrogen-sulphide containing feed gas and molecular-oxygen containing gas with a particulate oxidation catalyst in the presence of liquid elemental sulphur at a temperature in the range of from 120 to 160° C. The liquid elemental sulphur acts as an inert liquid medium that absorbs the heat generated by the exothermic oxidation reaction and thus prevents sulphur polymerisation and clogging of the catalyst or reactor due to an increase in sulphur viscosity.

In the selective oxidation process as described in WO2005/030638, a stream of liquid elemental sulphur, optionally containing catalyst particles, might be discharged from the selective oxidation reactor. Such elemental sulphur could for example be used as a binder in sulphur cement or in other sulphur cement products. It has, however, been found that in case the hydrogen sulphide containing feed gas comprises thiols, the elemental sulphur that is to be discharged from the selective oxidation reactor has an obnoxious smell. This smell is believed to be due to the presence of organic polysulphides that are formed from the thiols during the oxidation process. Also unconverted thiols remaining in the elemental sulphur might contribute to this smell. This smell disadvantageously limits the applicability of elemental sulphur obtainable from the selective oxidation process as described in WO2005/030638. Moreover, the presence of carbon-containing compounds, i.e. organic polysulphides or thiols, in the elemental sulphur also limits its applicability. For the application as raw material for sulphuric acid for example, elemental sulphur that is essentially free of carbon is needed.

### SUMMARY OF THE INVENTION

It has now been found that the obnoxious smell of elemental sulphur that contains organic polysulphides and/or thiols can be removed or diminished to an acceptable level by heating the elemental sulphur at a heating temperature in the range of from 360 to 700° C. at conditions at which a liquid elemental sulphur phase is maintained.

Accordingly, the invention provides a process for improving the smell of a stream of elemental sulphur comprising an organic polysulphide and/or a thiol, the process comprising the following step:
(a) heating the stream of liquid elemental sulphur comprising an organic polysulphide and/or a thiol at a heating temperature in the range of from 360 to 700° C., at a pressure that is sufficient to maintain a liquid elemental sulphur phase to obtain heat-treated elemental sulphur.

### DETAILED DESCRIPTION OF THE INVENTION

In the process according to the invention, elemental sulphur that contains an organic polysulphide and/or a thiol is heated at a heating temperature in the range of from 360 to 700° C. at a pressure that is sufficient to maintain the elemental sulphur in liquid phase.

Reference herein to an organic polysulphide is to a compound comprising a chain of sulphur atoms with an organic radical covalently-bound with an carbon atom to each end of the chain. Such compounds have the general molecular formula X—Sₙ—X′, wherein n is an integer with a value of at least 2, and X and X′ are, independently, an organic radical that is bound with a carbon atom to the sulphur chain Sₙ. Typically, X and X′ each are an alkyl radical.

Reference herein to a thiol is to a compound of the general molecular formula R—SH, wherein R is an organic radial, typically an alkyl radical such as methyl, ethyl, propyl, isopropyl or butyl, that is bound with a carbon atom to the sulphur atom.

It has been observed that during heating step (a) of the process according to the invention, carbon disulphide and hydrogen sulphide are formed. Without wishing to be bound to any theory, it is believed that the organic polysulphide(s) and/or thiol(s) present are converted to carbon disulphide and hydrogen sulphide if heated in the presence of liquid elemental sulphur.

In order to further improve the smell, hydrogen sulphide, carbon disulphide and other volatile compounds that may be formed during heating step (a) are preferably removed from the liquid elemental sulphur during heating step (a) by stripping. This may for example be done by leading a stripping gas through the liquid elemental sulphur during heating step (a). Any gas stream that is inert under the conditions of heating step (a) may suitably be used as stripping gas. Nitrogen or other inert gases are particularly suitable stripping gases.

Preferably, the stream of liquid elemental sulphur is kept at the heating temperature for at least 30 minutes, more preferably for a period in the range of from 1 to 20 hours. The optimum heating time will inter alia depend on the type(s) and amount of the polysulphide and/or thiol present and on the heating temperature. The optimum heating time may suitably be determined by measuring the hydrogen sulphide and carbon disulphide formation during heating step (a), for example by bubbling an inert gas through the liquid sulphur during heating step (a) and measuring the off-gas composition. As soon as the hydrogen sulphide and carbon disulphide formation drop to a negligible level, the heat treatment may be stopped.

The stream of liquid elemental sulphur is heated at a pressure that is sufficient to maintain a liquid elemental sulphur phase. The pressure will therefore depend on the heating temperature. Preferably, the pressure is in the range of from 3 to 50 bar (absolute).

The heating temperature is in the range of from 360 to 700° C. At temperatures above 700° C., the pressure required to maintain a liquid elemental sulphur phase is impracticably high. Below 360° C., the obnoxious smell is not sufficiently removed. Above 360° C., the smell is reduced to an acceptable level. Preferably, the heating temperature is in the range of from will 400 to 600° C., more preferably in the range of from 450 to 600. Above 450° no smell is observed any longer. Typically, a heating temperature of at least 500° C. will be needed in order to obtain heat-treated sulphur elemental sulphur that is substantially free of carbon-containing compounds.

The stream of elemental sulphur comprising an organic polysulphide and/or a thiol will typically comprise more than one polysulphide or thiol. The stream may be obtained from any source. A particularly suitable example of elemental sulphur that comprises organic polysulphides is the stream of elemental sulphur that may be withdrawn from a process of selective oxidation of hydrogen sulphide as is described in WO2005/030638. If the feed gas for the selective oxidation process comprises one or more thiols and the selective oxidation process is carried out in a liquid elemental sulphur phase at a temperature in the range of from 120 to 160° C., then the elemental sulphur that is discharged from the process comprises organic polysulphides, typically alkylpolysulphides, formed from the reaction of alkanethiols with elemental sulphur under the conditions applied.

Therefore, the process according to the invention preferably further comprises the following steps:
(b) supplying a gaseous feed stream comprising hydrogen sulphide and a thiol and a molecular-oxygen containing gas to a reaction zone comprising liquid elemental sulphur and particulate oxidation catalyst at a temperature in the range of from 120 to 160° C. to selectively oxidise the hydrogen sulphide to elemental sulphur; and
(c) discharging a stream of elemental sulphur comprising polysulphides from the reaction zone,

wherein stream of sulphur discharged from the reaction zone in step (c) is heated in step (a).

Process conditions and oxidation catalysts suitable for selective oxidation step (b) are described in more detail in WO02005/030638.

An alternative way to obtain a stream of elemental sulphur comprising an organic polysulphide and/or a thiol is by contacting a thiol-loaded purge gas from a thiol absorber with liquid elemental sulphur at a temperature in the range of from 120 to 160° C. Under these conditions, at least part of the thiols is converted into organic polysulphides. The conversion is preferably carried out in the presence of molecular oxygen and a particulate oxidation catalyst. The stream of elemental sulphur thus obtained comprises organic polysulphide and typically also unconverted thiol and may suitably be used in the process according to the invention.

The heat-treated sulphur elemental sulphur obtained with heating step (a) may be used for any known application of elemental sulphur. If the heat-treated sulphur elemental sulphur is substantially free of carbon-containing compounds, it may suitably be applied as raw material for sulphuric acid. Typically, a heating temperature of at least 500° C. will be needed in order to obtain heat-treated sulphur elemental sulphur that is substantially free of carbon-containing compounds. The heat-treated elemental sulphur is preferably used as binder in sulphur cement or a sulphur cement-aggregate composite. An advantage of the use of the heat-treated elemental sulphur as binder is that the presence of carbon-containing compounds is allowed and that thus not all organic polysulphides and/or thiols need to be removed.

Sulphur used as binder may be modified or plasticised in order to prevent allotropic transformation of the solid sulphur. Modified sulphur is typically prepared by reacting a portion of the sulphur with a sulphur modifier, also referred to as sulphur plasticiser. Modifiers are typically added in an amount in the range of from 0.05 to 25 wt % based on the weight of sulphur, usually in the range of from 0.1 to 10 wt %. A well-known category of sulphur modifiers, are olefinic compounds that co-polymerise with sulphur. Known examples of such olefinic sulphur modifiers are dicyclopentadiene, limonene, or styrene.

Sulphur cement is known in the art and at least comprises sulphur, usually in an amount of at least 50 wt %, and a filler. Usual sulphur cement fillers are particulate inorganic materials with an average particle size in the range of from 0.1 μm to 0.1 mm. Examples of such sulphur cement fillers are fly ash, limestone, quartz, iron oxide, alumina, titania, graphite, gypsum, talc, mica or combinations thereof. The filler content of sulphur cement may vary widely, but is typically in the range of from 5 to 50 wt %, based on the total weight of the cement.

A sulphur cement-aggregate composite is a composite comprising both sulphur cement and aggregate. Examples of sulphur cement-aggregate composites are sulphur mortar, sulphur concrete and sulphur-extended asphalt. Mortar comprises fine aggregate, typically with particles having an average diameter between 0.1 and 5 mm, for example sand. Concrete comprises coarse aggregate, typically with particles having an average diameter between 5 and 40 mm, for example gravel or rock. Sulphur-extended asphalt is asphalt, i.e. aggregate with a binder containing filler and a residual hydrocarbon fraction, wherein part of the binder has been replaced by sulphur.

Accordingly, the process according to the invention preferably further comprises the following steps:
(d) admixing the heat-treated elemental sulphur with at least any one of a sulphur cement filler, a sulphur modifier, or aggregate at a temperature at which sulphur is molten; and
(e) solidifying the mixture obtained by cooling the mixture to a temperature below the melting temperature of sulphur to obtain modified sulphur, sulphur cement or a sulphur cement-aggregate composite.

If only a sulphur modifier is admixed with the heat-treated sulphur in step (d), modified sulphur is obtained. If a sulphur cement filler and, optionally, a sulphur modifier is admixed, sulphur cement is obtained. If both a sulphur cement filler and aggregate are admixed, optionally together with a sulphur modifier, sulphur mortar or sulphur concrete are obtained. Preferably, the heat-treated elemental sulphur is admixed in step (d) with at least a sulphur cement filler and sulphur cement or a sulphur cement-aggregate composite is obtained in step (e).

In a preferred embodiment of the process according to the invention, the stream of liquid elemental sulphur that is heated in step (a) is the stream of elemental sulphur that is discharged from a reaction zone for selective oxidation step (b) and the heat-treated elemental sulphur obtained in step (a) is converted into modified sulphur, sulphur cement or a sulphur cement-aggregate composite according to steps (d) and (e). In a particularly preferred embodiment, the stream of elemental sulphur that is discharged from the reaction zone for selective oxidation step (b) comprises at least part of the particulate oxidation catalyst. The catalyst-comprising elemental sulphur is then heated in step (a). Thus, a heat-treated catalyst-comprising elemental sulphur is obtained that is converted into sulphur cement or a sulphur cement-aggregate composite according to steps (d) and (e). An advantage of this embodiment is that there is no need to separate elemental sulphur from the catalyst particles after selective oxidation step (b). As a consequence, very small catalyst particles may be used in selective oxidation step (b).

### EXAMPLES

The invention is further illustrated by means of the following non-limiting examples. Throughout the examples, flow rates of gaseous streams are expressed in Nl/hr, which stands for normal litres per hour. Normal litres are litres at conditions of standard temperature and pressure, i.e. 0° C. and 1 bar (absolute).

### Example 1

### Selective Oxidation of Hydrogen Sulphide in Liquid Sulphur

A slurry bubble column (internal diameter 20 mm) was loaded with 59 grams of elemental sulphur and 3.0 grams of small particles (average particle diameter is 10 μm) of iron oxide catalyst.

The iron oxide catalyst was prepared as follows. Silica extrudates having a surface area of 358 m²/g as measured by nitrogen adsorption (according to the BET method) and a pore volume of 1.34 ml/g as measured by mercury intrusion were provided with hydrated iron oxide. 100 grams of the silica extrudates were impregnated with 134 ml of a solution prepared from 28.6 grams of ammonium iron citrate (containing 17.5 wt % iron) and de-ionized water. The impregnated material was rotated for 90 minutes to allow equilibration. The material was subsequently dried at 60° C. for 2 hours, followed by drying at 120° C. for 2 hours and calcinations in air at 500° C. for 1 hour. The initial colour of the catalyst was black, but turned into rusty brown due to hydration of iron oxide. The resulting catalyst had a surface area of 328 m²/g, a pore volume of 1.1 ml/g and an iron content of 4.7 wt % based on the total catalyst weight.

The column was then pressurised with nitrogen to a pressure of 19 bar (absolute) and the temperature was raised to 140° C. A stream of feed gas (5.1 Nl/hr) comprising 39.2 vol % methane, 3.5 vol % carbon dioxide, 0.6 vol % hydrogen sulphide, 38 ppmv methanethiol and the remainder helium and air (0.6 Nl/hr) were bubbled through the slurry bubble column during 20 hours. After the 20 hours, the column was depressurised and the elemental sulphur was removed from the column. The catalyst particles were separated from the liquid elemental sulphur by filtration.

The liquid elemental sulphur thus obtained comprised alkylpolysulphides and had a smell that is typical for organic sulphides.

### Heat Treatment of Elemental Sulphur

In a quartz reactor tube with an internal diameter of 12 mm, 11 grams of elemental sulphur as obtained in the selective oxidation process described above was loaded. The tube was placed in an oven, pressurised with nitrogen to 6 bar (absolute) and heated to 140° C. to melt the sulphur. The temperature was then increased to 420° C. and the tube was maintained at that temperature for 2.5 hours. The temperature was then further increased to 465° C. and the tube was maintained at that temperature 3.5 hours. During the heat treatment, nitrogen was bubbled through the liquid sulphur at a rate of 1.24 Nl/hr. The reactor tube was then cooled to a temperature below the melting temperature of sulphur.

Analysis of the gaseous reactor effluent during the heat treatment showed a sharp peak in carbon disulphide and hydrogen sulphide formation upon heating to 420° C. The peak maximum was reached 30 minutes after the temperature of 420° C. was attained. Upon heating to 465° C., there was a new but smaller peak in carbon disulphide formation.

The heat-treated solid sulphur that was obtained did not have a sulphide smell anymore.

### Example 2

### Preparation of Polysulphide-Comprising Elemental Sulphur

Elemental sulphur comprising organic polysulphides was obtained as follows. In a 500 mL autoclave were loaded 300 grams of elemental sulphur and 20 grams of iron oxide catalyst particles. The catalyst particles were prepared as described in Example 1. The autoclave was closed and pressurised to 40 bar (absolute) using helium and nitrogen and heated at 132° C. A stream of pentane comprising 3.85 wt % butanethiol was supplied to the autoclave at a flow rate of 3.0 ml/hr. Helium (33 Nl/hr) and nitrogen (2 Nl/hr) were continuously bubbled through the reactor. After 90 hours, the autoclave was depressurised, the catalyst particles separated from the elemental sulphur by filtration and the butanethiol content of the elemental sulphur determined by Pyrolysis Combustion Mass spectrometric Elemental analysis (PCME analysis). This analysis showed that more than 99 wt % of the butanethiol supplied to the reactor was converted into organic polysulphides. The elemental sulphur had a pronounced sulphide smell.

### Heat Treatment of Elemental Sulphur

An amount of 10.4 grams of the elemental sulphur comprising organic polysulphides that was prepared as described above was loaded in a quartz tube with an internal diameter of 12 mm. The tube was placed in an oven, pressurised with nitrogen to 6 bar (absolute) and heated to 250° C. and maintained at that temperature for 20 hours. A flow of nitrogen of 2 Nl/hr was bubbled through the tube during the heat treatment. The gaseous reactor effluent was analysed by gas chromatography at 25 minute intervals. After 20 hours, the oven was switched off and the reactor was cooled by a flow of nitrogen to a temperature below the melting point of sulphur. The cooled reactor was depressurised and taken from the oven to assess the smell and colour of the elemental sulphur in the reactor.

Then the reactor with the same elemental sulphur was again placed in the oven and the procedure was repeated, but now at a heating temperature of 350° C. After assessment of the smell and colour of the heat treatment at 350° C., the procedure was repeated at 400° C., at 500° C. and at 600° C. In the table below the results of the smell assessment is given.

TABLE
Smell of elemental sulphur after different heat treatments

| Heating temperature | sulphide smell of |
|---|---|
| (° C.) | elemental sulphur |
| 250 | very strong |
| 350 | very strong |
| 400 | negligible |
| 500 | none |
| 600 | none |

1. A process for improving the smell of a stream of elemental sulphur comprising an organic polysulphide and/or a thiol, the process comprising the following step:
(a) heating the stream of liquid elemental sulphur comprising an organic polysulphide and/or a thiol at a heating temperature in the range of from 360 to 700° C., at a pressure that is sufficient to maintain a liquid elemental sulphur phase to obtain heat-treated elemental sulphur,
and further comprising the following steps:
(b) supplying a gaseous feed stream comprising hydrogen sulphide and a thiol and a molecular-oxygen containing gas to a reaction zone comprising liquid elemental sulphur and particulate oxidation catalyst at a temperature in the range of from 120 to 160° C. to selectively oxidise the hydrogen sulphide to elemental sulphur;
and
(c) discharging a stream of elemental sulphur comprising polysulphides from the reaction zone,
wherein the stream of sulphur discharged from the reaction zone in step (c) is heated in step (a).

2. A process according to claim 1, wherein volatile compounds are stripped from the liquid elemental sulphur during heating step (a).

3. A process according to claim 1, wherein the stream of liquid elemental sulphur is maintained at the heating temperature for a period of at least 30 minutes.

4. A process according to claim 1, wherein the stream of liquid elemental sulphur is heated at a pressure in the range of from 3 to 50 bar (absolute).

5. A process according to claim 1, wherein the heating temperature is in the range of from 400 to 600° C.

6. A process according to claim 1, further comprising the following steps:
(d) admixing the heat-treated elemental sulphur with at least any one of a sulphur cement filler, a sulphur cement modifier, or aggregate at a temperature at which sulphur is molten; and
(e) solidifying the mixture obtained by cooling the mixture to a temperature below the melting temperature of sulphur to obtain modified sulphur, sulphur cement or a sulphur cement-aggregate composite.

7. A process according to claim 6, wherein the heat-treated elemental sulphur is admixed in step (d) with at least a sulphur cement filler and sulphur cement or a sulphur cement-aggregate composite is obtained in step (e).

8. A process according to claim 1, wherein the stream of elemental sulphur discharged from the reaction zone in step (c) and heated in step (a) comprises particulate oxidation catalyst.

9. A process according to claim 2, wherein the stream of liquid elemental sulphur is maintained at the heating temperature for a period of at least 30 minutes.

10. A process according to claim 1, wherein the stream of liquid elemental sulphur is maintained at the heating temperature for a period in the range of from 1 to 20 hours.

11. A process according to claim 2, wherein the stream of liquid elemental sulphur is maintained at the heating temperature for a period in the range of from 1 to 20 hours .

12. A process according to claim 2, wherein the stream of liquid elemental sulphur is heated at a pressure in the range of from 3 to 50 bar (absolute).

13. A process according to claim 3, wherein the stream of liquid elemental sulphur is heated at a pressure in the range of from 3 to 50 bar (absolute).

14. A process according to claim 9, wherein the stream of liquid elemental sulphur is heated at a pressure in the range of from 3 to 50 bar (absolute).

15. A process according to claim 10, wherein the stream of liquid elemental sulphur is heated at a pressure in the range of from 3 to 50 bar (absolute).

16. A process according to claim 11, wherein the stream of liquid elemental sulphur is heated at a pressure in the range of from 3 to 50 bar (absolute).

17. A process according to claim 2, wherein the heating temperature is in the range of from 400 to 600° C.

18. A process according to claim 3, wherein the heating temperature is in the range of from 400 to 600° C.

19. A process according to claim 4, wherein the heating temperature is in the range of from 400 to 600° C.

20. A process according to claim 1, wherein the heating temperature is in the range of from 500 to 600° C.
